(19) United States
(12) Patent Application Publication
(10) Pub. No.: US 2017/0365458 A1
(43) Pub. Date: Dec. 21, 2017

(54) Adduct-Based System and Methods for Analysis and Identification of Mass Spectrometry Data

(71) Applicant: Woods Hole Oceanographic Institution, Woods Hole, MA (US)

(72) Inventors: James R. Collins, Seattle, WA (US); Bethanie R. Edwards, Honolulu, HI (US); Helen F. Fredricks, Rochester, MA (US); Benjamin AS Van Mooy, Falmouth, MA (US)

(21) Appl. No.: 15/613,187

(22) Filed: Jun. 3, 2017

Related U.S. Application Data
(60) Provisional application No. 62/345,175, filed on Jun. 3, 2016.

(51) Int. Cl. H01J 49/42; H01J 49/40; G01N 30/02; G01N 30/72; G06F 17/30; G01N 33/68

(52) U.S. Cl. CPC H01J 49/4215; G01N 30/72; G06F 17/30943; H01J 49/40; G01N 30/02; G01N 33/6848; G01N 2030/027; G01N 2030/025

(57) ABSTRACT

A system and method to screen a plurality of molecules in datasets obtained from mass spectroscopy, including selecting and receiving at least one dataset of mass spectral data, and selecting customizable m/z mass tolerance peaks to assign initial compound assignments from at least one adduct ion hierarchy database for at least one compound having a parent molecule. Adduct ion hierarchy screening is applied to at least a portion of the dataset, wherein selected dataset features are tested to determine if they represent the most abundant expected adduct of the parent molecule class and if the expected adduct assignment hierarchy are present in the dataset.

### CROSS REFERENCE TO RELATED APPLICATION

This application claims priority to U.S. Provisional Application No. 62/345,175 filed on 3 Jun. 2016. The entire contents of the above-mentioned application is incorporated herein by reference.

### FIELD OF THE INVENTION

This invention relates to an improved mass spectra analysis method; more particularly, the invention relates to high accuracy, hierarchy-based screening and identification of molecules in data obtained from mass spectrometry.

### BACKGROUND OF THE INVENTION

Mass spectrometry (MS) is an analytic technique used to detect, identify and quantify molecules present in a given sample. Many organic and inorganic substances can be ionized and can therefore be analyzed by this technique. MS is an extremely versatile and useful tool, which has been used in a wide range of applications, including screening newborns for metabolic disorders, comparing cellular protein expression, determining food's mineral bioavailability, analyzing drug metabolism in a patient, identifying pesticides, toxins and pollutants in the environment, and monitoring biomarkers for a biological response, like oxidative stress, for example.

Although MS has many uses, it generates large, difficult-to-analyze mass-to-charge (m/z) datasets. MS breaks down samples with an ion source (e.g. a stream of electrons) to create charged fragments, or adduct ions. Some of these charged fragments will in turn breakdown or be further ionized. Because of the large number of variables and restrictions, such as even vs odd electron species, ion mode, and degree of fragmentation, MS datasets are difficult to analyze and annotate.

However, many ionizable molecules form consistent, diagnostic patterns of adduct ions when fragmented during MS. Computer software programs can attempt to identify m/z intensity peaks with specific ions and their parental molecules, but very few such program exist, and those that do are limited in scope. One such software program, LipidSearch by ThermoFisher Scientific, can only identify a subset of oxidized lipids. Furthermore, when compared together, the currently available MS annotation software programs often do not identify the same molecules in a given sample.

One critical need is to identify reactive oxygen species (ROS), which represent a persistent source of stress in virtually all biological systems. The negative cellular effects of ROS include protein damage, mutation of DNA, and lipid peroxidation. The profound effects of oxidative stress have been extensively documented in the lipids of mammals and other organisms.

Furthermore, oxidative stress and ROS induce equally significant and wide-ranging remodeling of cell lipid profiles (lipidomes) in both terrestrial and marine organisms. These ROS can act through a variety of enzymatic and abiotic mechanisms to produce a broad and heterogeneous suite of lipid products whose bioactivity and diversity make them ideal as molecular biomarkers. These products include both oxidized intact polar lipids (ox-IPL; e.g., oxidized phospholipids) as well as oxylipins, the smaller, direct derivatives of fatty acids. Lipid biomarkers (both oxidized and unoxidized) can be used to characterize the effects of ROS in humans from cancer and other diseases such as atherosclerosis; in the marine environment, lipids can be used to diagnose various sources of biological and abiotic stress, including those imposed by nutrient limitation and viral infection. The potency and specificity that make lipids useful as biomarkers of oxidative stress also support their function as bioactive “infochemicals.” In the ocean, for example, oxylipins have been shown to regulate different interspecific interactions among marine microbiota and the metabolism of sinking marine particles by heterotrophic bacteria.

For several reasons, there exist few comprehensive methods to screen, identify, and annotate large numbers of these oxylipins and oxidized lipids alongside the many unoxidized lipids from which they originate. Oxylipins, like their parent lipids, have a wide diversity of structures and biochemical functions. They can be produced enzymatically or abiotically, often occurring in very low abundance relative to their intact polar lipid (IPL) precursors. Finally, unique and tailored computational strategies are required to analyze the large volumes of data necessary for comprehensive lipidomics or metabolomics.

The limited number of analytical strategies developed specifically to assess the effects of oxidative stress on the lipidomes of humans and mammals have generally focused on traditional oxylipins, such as hydroperoxy, hydroxy, epoxy, oxo, and ketol fatty acids, while ignoring most molecular precursors and intermediates. For example, direct-infusion mass spectrometry has been used to identify select oxylipins simultaneously with their unoxidized IPL and ox-IPL precursors in the model plant Arabidopsis thaliana, but these studies used manual, not automated, computer-based data analysis methods to examine oxidation of compounds containing only C₁₆ and C₁₈ fatty acids. Some studies employed a shotgun approach to identify oxidized lipids in rat cardiomyocytes, but their analysis was limited only to intact carbonylated phospholipids.

The commercial LipidSearch software (Thermo Scientific) can identify some oxidized lipids using MS/MS fragmentation spectra, but this capability does not extend to oxylipins derived from fatty acids. Furthermore, there are no comprehensive methods to screen, identify and annotate diverse molecule classes, not just specially prepared lipid samples.

One approach recognized by the present inventors is to take advantage of adduct ion cascades that reproducibly form when a potential biomarker molecule is fragmented after ionization. Most ionizable molecules (M) form a series of fragment ions in a set order of abundance (e.g. phosphatidylcholine in positive ion mode, reproducibly produces an adduct ion hierarchy of [M+H]+>[M+Na]+>[m+NH4+ACN]+>[M+2Na—H]+>[M+K]+). Each adduct ion has specific and identifiable m/z ratio, and taken together can predict the presence of the parent molecule. Therefore, there is an unsatisfied need for systems and methods to identify molecules in large MS datasets. A rules-based screening approach that takes advantage of adduct ion hierarchies formed during MS ionization that can identify any ionizable molecule with the aid of standardized reference databases will greatly advance the field of MS analyzation and annotation.

### SUMMARY OF THE INVENTION

Accordingly, it is an object of the present invention to provide a system and method of automatically identifying ionizable molecules in large, high-mass-accuracy high performance liquid chromatography and/or ionization mass spectrometry datasets.

This invention features a method of screening a plurality of molecules in datasets obtained from mass spectroscopy, including selecting and receiving at least one dataset of mass spectral data, and selecting customizable m/z mass tolerance peaks to assign initial compound assignments from at least one adduct ion hierarchy database for at least one compound having a parent molecule. The method also includes applying adduct ion hierarchy screening to at least a portion of the dataset, wherein selected dataset features are tested to determine if they represent the most abundant expected adduct of the parent molecule class and if the expected adduct assignment hierarchy are present in the dataset.

In some embodiments, the method further includes pre-processing the dataset including aggregating the dataset into subsets based on at least one feature, and applying a first screening criteria to identify and remove secondary isotypes in the subsets. In certain embodiments, the method includes applying a second screening criteria of retention time to at least one subset based on a relationship of retention time of the dataset features as compared with a predefined retention time window of the compound assignment's parent molecule class. In some embodiments, the method includes applying a fourth screening criteria to identify and annotate isomer and isobars in the subset. In certain embodiments, the method further includes assigning one or more identifications and annotations to each feature in the subset. In one embodiment, preprocessing the dataset includes at least one of feature detection, retention time correction, peak grouping, m/z, secondary isotope identification, and a combination thereof. In certain embodiments, the feature is selected from retention time, acyl carbon number, peak, and a combination thereof. In one embodiment, the first screening criteria comprises identifying and excluding secondary isotopes. In another embodiment, the method further includes applying a third screening criteria to the subset to exclude one or more specific molecules, specific chemical moieties or molecules containing a specific number of one or more chemical moieties, in a customizable manner.

In some embodiments, the method further includes formatting the screened subset such that it will be analyzable by additional software on a computer device. In one embodiment, the method further includes performing statistical analysis on the subset after adduct ion hierarchy screening is applied. In some embodiments, the method further includes exporting the screened subset to a common file format readable by additional software on a computer device. In certain embodiments, the method annotates the resulting subset with codes demarking the degree to which the assignment complies with the hierarchy screening criteria. In one embodiment, at least one adduct ion hierarchy database is generated in positive ion mode. In another embodiment, at least one adduct ion hierarchy database is generated in negative ion mode. In a number of embodiments, the method includes generating the dataset utilizing at least one additional chemical that is added to an eluent to which the molecules are exposed at least prior to the mass spectroscopy.

In certain embodiments, the method further includes preparing at least one adduct ion hierarchy database, and selecting that database for screening the dataset. In one embodiment, the method further includes generating adduct ion hierarchy databases from empirical data produced from standardized parent molecules that have undergone ionization and measurement by mass spectrometry. In one embodiment, the method further includes ranking the databases by adduct ion ranking. In a number of embodiments, the mass spectrometry data is selected to include at least one of liquid chromatography-mass spectrometry data, gas chromatography-mass spectrometry data, Fourier transform mass spectrometry data, direct infusion mass spectrometry data, capillary electrophoresis mass spectrometry data, ion mobility shift mass spectrometry data, desorption electrospray ionization mass spectrometry data, nanostructure initiator mass spectrometry or matrix assisted mass spectrometry data. In some embodiments, the method further includes generating a confidence value for the identifications assigned to each feature.

The invention also features a system for screening a plurality of molecules in mass spectroscopy datasets, the system comprising a processor programmed to execute at least one of the above methods. The invention further features a computer program for screening a plurality of molecules in mass spectroscopy datasets, the program comprising at least one of the above methods, wherein said program is executed on a computer device.

This invention may also be expressed as a new, hierarchy-based screening system and method for automatically identifying any ionizable molecule in large, high-mass-accuracy high performance liquid chromatography/electrospray ionization mass spectrometry (HPLC-ESI-MS) datasets. This inventive system and method is implemented in one embodiment as a software package for computer devices and integrates with the existing software packages for additional functionality. The software is centered around a novel screening methodology that exploits the unique tendency of ionizable molecules to form adduct ions in consistent, diagnostic patterns of abundance that remain relatively consistent across sample types (e.g., phosphatidylcholine in positive ion mode, reproducibly produces a adduct ion hierarchy of [M+H]+>[M+Na]+>[M+NH4+ACN]+>[M+2Na—H]+>[M+K]+). The software is able to resolve conflicting compound assignments, examine differential expression of compounds across experimental treatments, discover and identify potential ox-IPL and oxylipin biomarkers, and identify potential isomers and isobars when applied to mass spectrometry datasets from mutant marine diatom designed for oxidative stress studies.

In one embodiment, the software annotates each compound assignment with a confidence score, allowing the user to define subsets for further analysis. While the software has been used to identify biomarkers in a marine microorganism, it will apply to any HPLC-ESI-MS dataset where the user expects the relative proportions of the various adduct ions of each analyte to remain constant across samples. The software is designed to be used to analyze data acquired via HPLC-ES-MS, FT-ICR-MS, or, when sufficient allowances are made for mass resolution, a Quadrupole-time-of-flight (Q-TOF) instrument.

### BRIEF DESCRIPTION OF THE DRAWINGS

The drawings described herein constitute part of this specification and include exemplary embodiments of the inventive system and methods, also referred to as the software, which may be further embodied in various forms. It is to be understood that in some instances, various aspects of the invention may be shown exaggerated or enlarged to facilitate an understanding of the invention. One or more drawings and Tables can be generated and presented on a user interface. In what follows, preferred embodiments of the invention are explained in more detail with reference to the drawings, in which:

FIGS. 1A-1D are schematic flow charts of preparation, screening, and annotation of HPLC-MS Lipid Data according to the present invention, with FIG. 1A showing pre-processing and feature detection, FIG. 1B showing compound assignments and initial screening criteria, FIG. 1C showing application of core adduct ion hierarchy screening rules, and FIG. 1D showing isobar and isomer detection, annotation, and optional outputs;

FIG. 2A is a schematic graph showing m/z on the y-axis and corrected retention time in minutes on the x-axis;

FIG. 2B is a schematic chart of distribution by lipid class of high-confidence assignments depicted in FIG. 2A, with ellipse size representing the number of compounds identified in each class and treatment;

FIGS. 3A-3C show remodeling of the Phaeodactylum tricornutum lipidome after 24 h, as visualized from data analyzed with the software system, with FIG. 3A being a heatmap showing relative abundances across two treatments (0 and 150 μM H₂O₂) of all IPL, ox-IPL, and TAG identified with high confidence, with each row (N=896) representing a different compound identified from the database;

FIG. 3B is heatmap detail, showing changes in the most abundant (N=40) moieties of monogalactosyldiacylglycerol (MGDG), a lipid typically localized to the chloroplast;

FIG. 3C is a fraction of total peak area identified as triacylglycerol (TAG) at three timepoints during the experiment. Error bars are ±SD of two replicates;

FIG. 4. Is an extracted ion chromatogram (m/z 500-1500; positive ion mode) from a P. tricornutum sample treated with 150 μM H₂O₂;

FIGS. 5A-13B are charts showing oxidized and intact species of nine classes of lipid identified in the P. tricornutum dataset after 24 hours in (A) the control (0 μM H₂O₂) and (B) 150 μM H₂O₂ treatments, for species of, respectively, DGCC (FIGS. 5A-5B), DGDG (FIGS. 6A-6B), DGTS & DGTA (FIGS. 7A-7B), MGDG (FIGS. 8A-8B), PC (FIGS. 9A-9B), PE (FIGS. 10A-10B), PG (FIGS. 11A-11B), SQDG (FIGS. 12A-12B), and TAG (FIGS. 13A-13B), specifically FIGS. 5A and 5B are charts showing DGCC control and treatments, respectively, FIGS. 6A and 6B are charts showing DGDG control and treatments, respectively, FIGS. 7A and 7B are charts showing DGTS & DGTA control and treatments, respectively, FIGS. 8A and 8B are charts showing MGDG control and treatments, respectively, FIGS. 9A and 9B are charts showing PC control and treatments, respectively, FIGS. 10A and 10B are charts showing PE control and treatments, respectively, FIGS. 11A and 11B are charts showing PG control and treatments, respectively, FIGS. 12A and 12B are charts showing SQDG control and treatments, respectively, and FIGS. 13A and 13B are charts showing TAG control and treatments, respectively; and

FIG. 14 is an expanded version of the heatmap in FIG. 3A, showing remodeling of the Phaeodactylum tricornutum lipidome after 24 h.

### BRIEF DESCRIPTION OF THE TABLES

Table 1. Quality control samples of known composition analyzed by the inventive software system.

Table 2. Progressive screening and annotation of the P. tricornutum dataset using xcms, CAMERA, and the inventive software system.

Table 3. Database dimension and ranges of structural properties considered for each lipid class.

Table 4. Relative abundances, by rank, for adduct ions of lipid and oxylipin species in the database for Example 1.

Table 5. Pigment abbreviations used in the software system's database.

Table 6. Retention time window criteria for various compounds and compound classes.

Table 7. xcms, CAMERA and inventive software system settings used in analysis of the P. tricornutum dataset.

Table 8. Evaluation of method performance using IPL standards and alternative software systems for feature detection and chromatographic alignment.

Table 9. Annotation of isomers and isobars in screened P. tricornutum dataset.

Table 10. List of groups of P. tricornutum lipidome components determined by similarity profile analysis on 0 μM and 150 μM H₂O₂ treatments at 24 hours.

Table 11. Molecular Characteristics of IPL, ox-IPL, and TAG observed in P. tricornutum after 24 hours.

Table 12. Examples of isomers and isobar annotation for the P. tricornutum dataset.

Table 13. Example of confidence codes available for the system to annotate compound assignments.

### DETAILED DESCRIPTION OF THE PREFERRED EMBODIMENTS

The described features, advantages, and characteristics may be combined in any suitable manner in one or more embodiments. One skilled in the relevant art will recognize that the system may be practiced without one or more of the specific features or advantages of a particular embodiment. In other instances, additional features and advantages may be recognized in certain embodiments that may not be present in all embodiments.

This invention may be accomplished by a system and/or method of screening a plurality of molecules in datasets obtained from mass spectroscopy, including selecting and receiving at least one dataset of mass spectral data, optionally pre-processing the dataset including aggregating the dataset into subsets based on at least one feature, and optionally applying a first screening criteria to identify and remove secondary isotypes in the subsets. The method further includes selecting customizable m/z mass tolerance peaks to assign initial compound assignments from at least one adduct ion hierarchy database for at least one compound having a parent molecule, and optionally applying a second screening criteria of retention time to at least one subset based on a relationship of retention time of the dataset features as compared with a predefined retention time window of the compound assignment's parent molecule class. The method also includes applying adduct ion hierarchy screening to at least a portion of the database, such as to at least the subset, wherein selected dataset features are tested to determine if they represent the most abundant expected adduct of the parent molecule class and if the expected adduct assignment hierarchy are present in the dataset.

### Design and Scope of Adduct Ion Databases.

The inventive software system draws compound assignments from customizable databases that contain structural and adduct ion abundance data for any molecule that generates a series of reproducible ionization reactions, including nonpolar lipids, IPL, ox-IPL, and oxylipins (Table 3; Table 5). Each database entry represents a different adduct ion of a potential analyte; because analytes present differently in positive and negative ionization modes, separate onboard databases have been generated for compound identification in each mode and custom databases can be supplied by the user for each mode. In one embodiment, the software system includes at least one default database that contain entries for 14,068 unique compounds, some of them particular to marine algae (Table 3; Table 5). Alternatively, some embodiments allow the user to generate their own databases. The use of onboard databases is one distinguishing feature of the present software system over other conventional software packages that rely exclusively on external databases.

### Adduct Ion Database Generation.

Databases are created in the software system by pairing empirical data with an in silico simulation. The onboard databases present in some embodiments were generated by first calculating exact masses for various triacylglycerols (TAG), free fatty acids (FFA), polyunsaturated aldehydes (PUA), and molecules belonging to eight different classes of intact polar diacylglycerol (IP-DAG). Within each of these classes, the masses of a wide range of possible structures having fatty acid (FA) moieties of different acyl chain length, unsaturation, and oxidation were calculated (Table 3). The exact masses for several photosynthetic pigments common to the marine environment were also included (Table 3; Table 5). Molecules and adduct ions are further identified by the “sum composition” of their constitutive double bonds and acyl carbon atoms in each compound (e.g., PC 34:1, rather than PC 16:0-18:1).

### Determination of Relative Abundances of Adduct Ions for Inclusion in Databases.

Different chemical structures (e.g. having a different shape and/or polarity) cause different molecules to interact in distinct and reproducible ways with the LC/MS eluent chemicals, including different chemicals that may be added to the LC/MS eluents. The resulting adducts are ranked based on the overall proportion of each type of adduct ion present for the specified conditions. During database generation, the system uses empirical data for the LC/MS adduct ion(s) typically formed by each compound's parent, either from default databases such as shown in Table 4, or from user-supplied databases. Multiple entries of adduct ions for each parent compound, also referred to as a parent molecule, are entered into the database, each entry representing one commonly-formed adduct ion. The ranking of adduct ions form the basis for the hierarchy-based screening of compound assignments by systems according to the present invention. In a preferred embodiment, authentic standards for representative compounds were used to confirm any onboard databases.

In many embodiments, a series of tables can be used with the software system to define additional analytes and adducts beyond those which are included in the onboard databases. For each new molecule or molecule class, the software system requires (1) the elemental composition of the new molecule or parent molecule of the new molecule class, (2) a tabulation of expected adducts (defining, as necessary, any new adducts), (3) empirical adduct hierarchy data for any new adducts, and (4) if applicable, the ranges of acyl carbon atoms, double bonds, and oxidization states for which entries are to be generated.

### Lipidomics Workflow Based on Xcms, CAMERA, and the Software System.

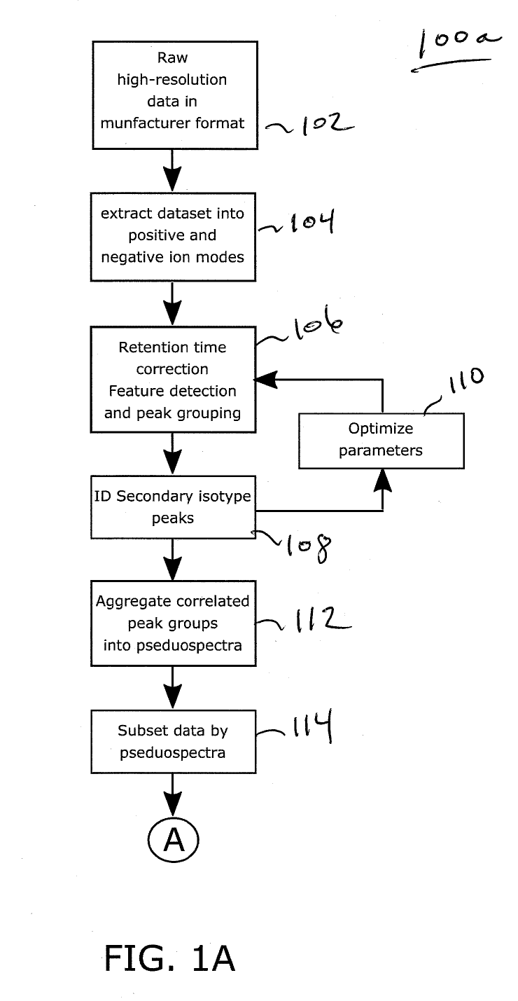

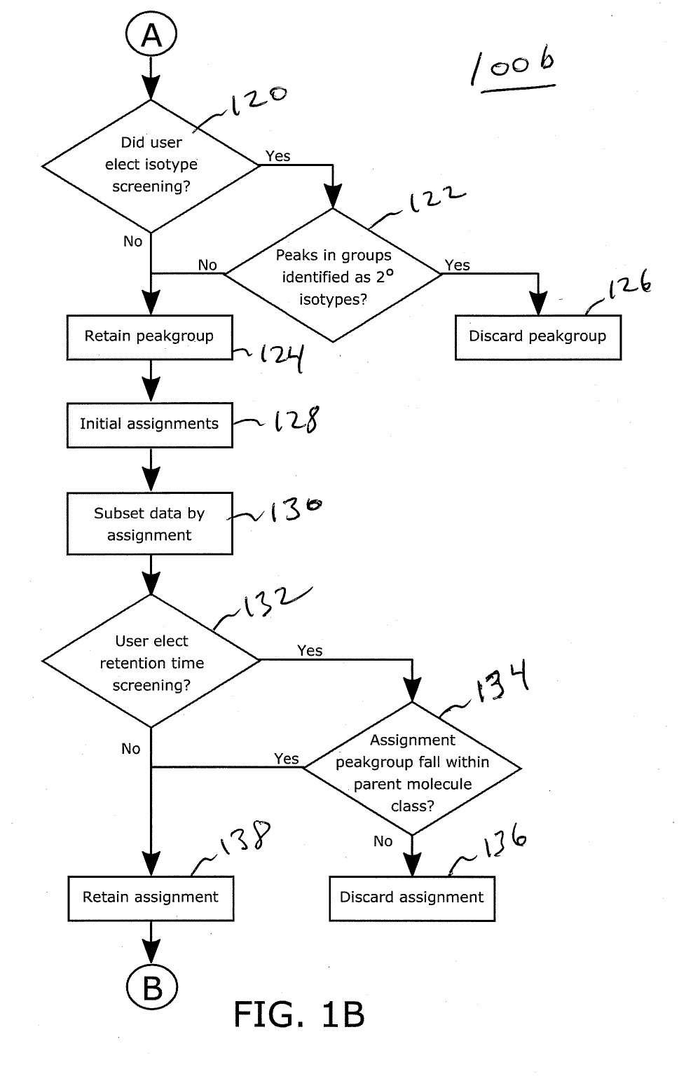

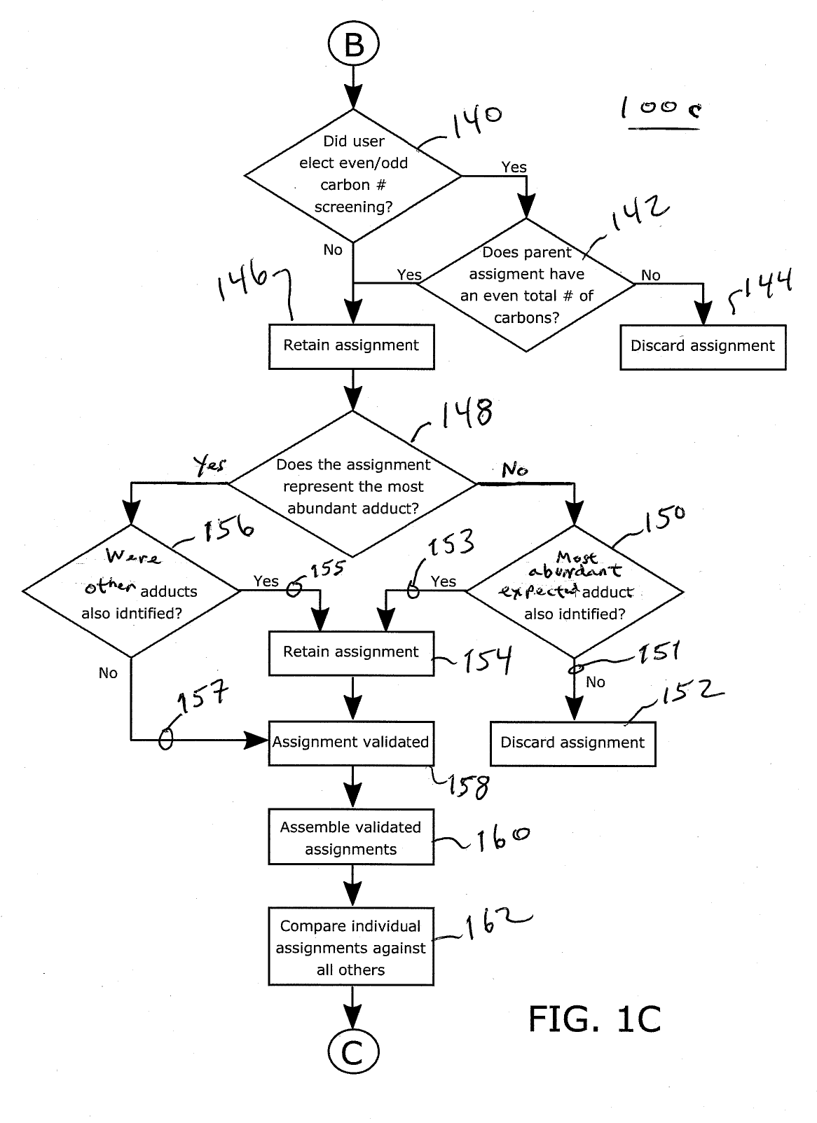

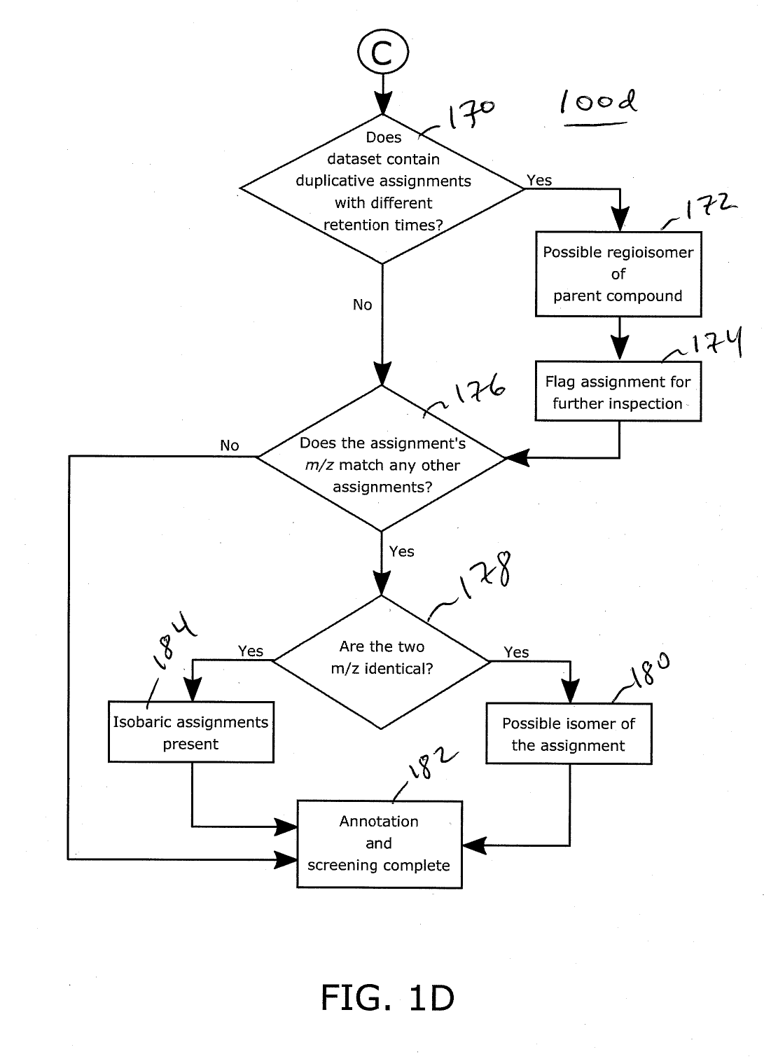

Data is supplied to the inventive software system, step 102, FIG. 1A, and designated as positive or negative ion mode, step 104, and extracted into separate inputs. Two ions are considered isobaric when the underlying features have different exact masses but the m/z difference is less than the acquisition instrument's demonstrated mass accuracy (e.g. 2.5 ppm). Once data files have been converted and extracted, steps 102-104, the software system pre-processes the data for selected feature detection and peak grouping, steps 106-110. Chromatographic alignment and retention time correction can also be made. Identification of pseudospectra, and identification and selection of features representing possible secondary isotope peaks are made in steps 108, 112 and 114 in this construction.

### Database Assignments and Progressive Screening Using Orthogonal Criteria.

In many embodiments, after pre-processing, FIG. 1A, the steps of screening and annotation are performed according to the workflow depicted for flowcharts 100b-100d, FIGS. 1B-1D. After pre-processing & feature detection as shown in flowchart 100a, FIG. 1A, compound assignments and initial screening criteria are made as shown in flowchart 100b, FIG. 1B. Core adduct ion hierarchy screening rules are applied in flowchart 100c, FIG. 1C, and then isobar and isomer detection, annotation, and optional outputs are made in flowchart 100d, FIG. 1D.

First, initial compound assignments are applied to features from the database using a narrow, customizable m/z mass tolerance specified by the user, step 120, FIG. 1B. A series of optional orthogonal screening criteria can be further applied to the features and their assignments, steps 124, 128 and 130. Users may exclude from the dataset any features representing secondary isotope peaks, step 122; the presence of these features is a significant drawback of current mass spectrometry software algorithms. In many embodiments, the inventive software system is designed to exclude these secondary isotype peaks, step 126, rather than merge the elements of each feature's isotopic envelope into a single parameter.

Next, the software system screens the feature's retention time against a retention time “window” defined for the accompanying assignment's parent lipid class, steps 132 and 134. Many preferred embodiments include a set of default retention time window data (Table 6) for the chromatographic conditions described herein. Additional, optional filters can be applied in some embodiments to exclude assignments, steps 134 and 136, of specific molecules, for example: IPL, ox-IPL, FFA, and PUA that may contain one or more specific properties (e.g. an odd total number of acyl carbon atoms). Some embodiments would apply filters specific to data derived exclusively from eukaryotic origin, because non-acetogenic fatty acid synthesis is confined almost exclusively to bacteria and archaea, allowing for improved and faster analysis of the mass spectrometry data.

After applying the above initial optional criteria and deciding which assignments to retain, step 138, FIG. 1B, the software system then screens each assignment using adduct ion hierarchy data according to the present invention, such as by application of core adduct ion hierarchy screening rules shown in flowchart 100c, FIG. 1C, and Table 4. This inventive screening is the primary orthogonal filter that eliminates confounding secondary isotopes and unassigned lipid-extractable features still remaining in the dataset. The software system uses a series of customizable rules to compare the relative abundance ranks of sets of adduct ion assignments that have the same parent compound.

In one construction, the system determines whether the user elected even/odd carbon number screening, step 140, FIG. 1C, such as even or odd acyl carbon numbering or other portion of a fatty acid, for example. If yes, it is determined whether the parent compound of the assignment has an even total number of carbon atoms, step 142. If no, the assignment is discarded, step 144; if yes, the assignment is retained, step 146, as a specific assignment.

Continuing the explanation of one construction of the present invention, after retaining the specific assignment, the system then determines if the assignment represents the most abundant adduct, step 148. More particularly, is the adduct represented by this assignment the most abundant expected adduct of the parent compound? If not, it is determined whether the most abundant expected adduct of the parent compound was also identified in its pseudo-spectrum, step 150. If not, the assignment is discarded, step 152, and the terms “C4” or “C5” may be designated as value 151 in certain constructions. The term “value” is also referred to herein as “code” or “signal”. An example of codes utilized in one construction according to the present invention is provided in Table 13. Returning to FIG. 1C, if “yes” is found in step 150, the assignment is retained, step 154, and the terms “C2a” or “C2b” may be designated as value 153 for this assignment.

The code or value C2a indicates that the adduct ion hierarchy for the parent compound is completely satisfied, that is, the pseudo-spectrum contains peak-groups representing every adduct ion of the compound of greater theoretical abundance than the least abundant adduct ion present. The code or value C2b indicates that the adduct ion of greatest theoretical abundance and some lesser adduct ion is present, but adduct ions of intermediate abundance are not observed. These “annotation code” values, when designated, assist the system and/or the user in evaluating assignment confidence during subsequent data analysis. In other words, in some embodiments the software system annotates the resulting assignment data using simple codes that indicate the degree to which the assignment complies with the hierarchy rules. Assignments that fail the adduct ion hierarchy screening criteria are excised from the dataset and all remaining assignments in the dataset are then pooled.

If the system determines in step 148, FIG. 1C, that the adduct represented by the assignment is the most abundant expected adduct of the parent compound, then it is determined, step 156, if any other adducts of the same parent compound were also identified in the pseudo-spectrum. If yes, the assignment is retained, step 154, and the terms “C2a” or “C2b” may be designated as code or value 155 for this assignment. If not, then the assignment is validated, step 158 and the term “C1” may be designated as value 157. The validated assignments are then assembled from all pseudo-spectra, step 160, and compared individually against all others, step 162.

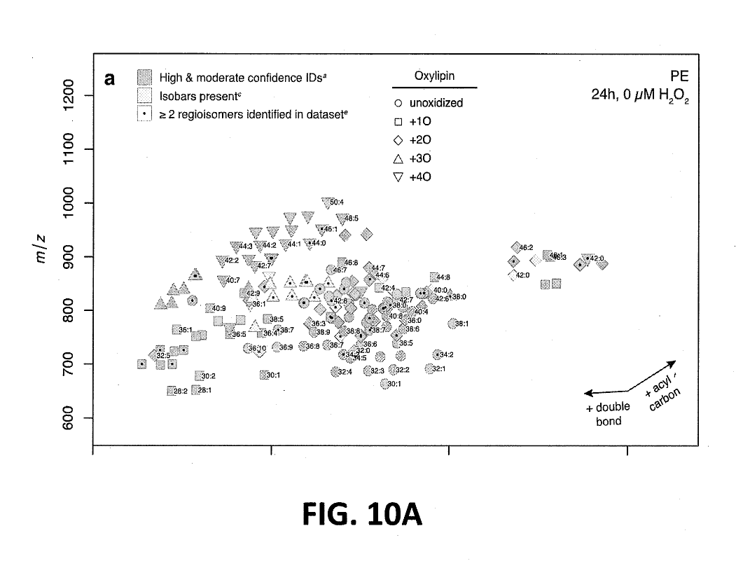

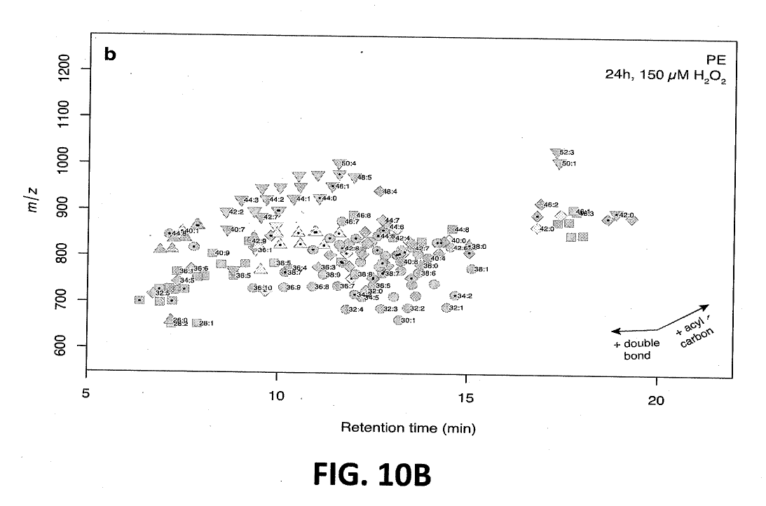

In many preferred embodiments, additional rules-based screening is performed on the pooled data to identify and annotate possible isomers and isobars, such as isomer and isobar detection and annotation represented by flowchart 100d, FIG. 10. For example, does the dataset contain duplicative assignments with different retention times as determined in step 170. In other words, does the dataset contain additional instances of the same parent compound at different observed retention times. If “yes”, possible regio-isomers of the parent compound may be present, step 172, and the assignment is flagged, step 174, by a code such as “C3r”. Isomers may be cross-referenced to facilitate further inspection, and the logic proceeds to step 176, as it does if step 170 determines “no” duplicative assignments.

In step 176, FIG. 1D, the system determines whether the assignment's m/z falls within match ppm of m/z of any other assignments in the database. If yes, then possible isomer of the assignment is present, step 180. The assignment can be flagged with the code “C3f”, and isomers are cross-referenced to facilitate further inspection. Alternatively, if the two m/z are determined to be identical in step 178, isobaric assignments are present, step 184, and the assignment can be flagged with a code “C3c”. Once annotation is made in step 180 or in step 188, then annotation and screening for that assignment is completed, step 182. Similarly, if there is no m/z match found in step 176, the logic proceeds to step 182.

Codes can be applied to identify positional or regio-isomers, functional structural isomers, or isobars. The software system can apply one or more codes to a given assignment as long as the criterion for each is satisfied. Upon completion of screening, some embodiments of the software system produces an annotated dataset, while other embodiments of the software system produce computer code containing the annotated dataset for additional computer software. Some embodiments of the software system will then perform statistical analysis on the final matrix of compound assignments. Some embodiments will export the final results to a common file format for external analysis.

### Example 1

### Model Dataset Used to Demonstrate the Software System.

Oxidative stress is an imbalance between reactive oxygen species and an organism's ability to detoxify the reactive molecules and repair any damage caused by the reactive molecules. Oxidative stress is believed to play important roles in the pathogenesis of many human diseases, including cancers, autism, infections and Parkinson's disease, to name a few. Understanding and measuring an organism's level of oxidative stress is an important step of identity and treating human disease before it can detrimentally impact the individual.

One use of the present system is to examine the effect of oxidative stress on a model algal lipidome, providing for a better understanding of the mechanisms and effects of oxidative stress. The present software system takes mass spec data collected from cultures of a mutant strain of the marine diatom Phaeodactylum tricornutum, which was designed for studies of oxidative stress. In this specific example, a strain of P. tricornutum (CCMP2561; Provasoli-Guillard National Center for Marine Algae and Microbiota) was genetically modified to express a reduction-oxidation sensitive green fluorescent protein (roGFP) at different locations within the cell. Cultures of the transformants were treated with three concentrations of H₂O₂ (0, 30, and 150 μmol L⁻¹) to evaluate the effects of peroxidation. The software utilized in this Example can be found in one or both of the following code repositories of Github at https://github.com/vanmooylipidomics/LOBSTAHS and Bioconductor at http://bioconductor.org/packages/release/bioc/html/LOBSTAHS.html and are incorporated herein by reference.

### Sample Collection and Extraction.

In this example, duplicate samples for lipid analysis were collected from each treatment at 4 hour, 8 hour, and 24 hour timepoints. Two procedural blanks were also collected. Sample material was collected by vacuum onto 0.7 μm pore size glass fiber filters (GF/F), which were snap frozen in liquid nitrogen and then stored at −80° C. until thawed for extraction. Extraction was performed using a modified Bligh and Dyer method described in Popendorf et al.; an internal standard (dinitrophenyl-phosphatidylethanolamine, DNP-PE) and a synthetic antioxidant (butylated hydroxytoluene, BHT) were added at time of extraction. Lipid extracts were transferred to 2 mL HPLC vials, topped with argon, and stored at −80° C. prior to analysis. All chemicals used in sample extraction and chromatography were LC/MS grade or higher. Where used, water was obtained from a Milli-Q system without further treatment (EMD Millipore, Billerica, Mass., USA).

### HPLC-ESI-MS Analysis.

Samples from the P. tricornutum dataset were analyzed by HPLC-ESI-MS using a modification of the method described in Hummel et al. Lipid extracts were evaporated to near dryness and reconstituted in a similar volume of 7:3 acetonitrile:isopropanol. Headspace was filled with argon to minimize further oxidation. For HPLC analysis, an Agilent 1200 system (Agilent, Santa Clara, Calif., USA) comprising temperature-controlled autosampler (4° C.), binary pump, and diode array detector, was coupled to a Thermo Exactive Plus Orbitrap mass spectrometer (ThermoFisher Scientific, Waltham, Mass., USA). Chromatographic conditions, electrospray ionization source settings, MS acquisition settings, and procedures used for calibration of the mass spectrometer are described in the Supporting Information. Using authentic standards and two independent methods for MS feature detection, we determined the average relative mass uncertainty of the Exactive was <0.2 ppm (Table 1; Table 8).

Analysis of P. tricornutum Data Using the Software System.

The software system was then used to identify and annotate lipidome components in the positive ionization mode data. In this example, the embodiment used the R package IPO to optimize settings for several xcms functions, and a 2.5 ppm mass uncertainty tolerance was used to obtain database matches in the software system. In other embodiments, the IPO functionality will be included in the software system. Using the annotated output obtained from the software system, the relative abundances of lipidome constituents present in the 0 and 150 μM H₂O₂ treatments at 24 h was calculated. Statistical techniques were used to identify biomarkers of oxidative stress. Unless otherwise noted, the analysis was restricted to only “high confidence” assignments; these were assignments without structural isomers or isobars given codes of C1 or C2a according to the logic in FIG. 1C as described above. The specific settings used in the xcms, CAMERA, and software packages, details of statistical methods, and links to the software code is referenced in its entirety in Collins, J. R., Edwards, B. R., Fredricks, H. F., and Van Mooy, B. A. S., “LOBSTAHS: A novel lipidomics strategy for semi-untargeted discovery and identification of oxidative stress biomarkers”, Anal. Chem. 2016, 7154-7162, Vol. 88, Amer. Chem. Soc.

Screening and Annotation of P. tricornutum Data in the Software System.

In this example the software system identified 21,869, or 6.4%, of the 340,991 mass spectral features initially detected in the dataset. Sequential application of the various screening criteria allowed for the exclusion of features from the dataset based on specific characteristics (Table 2). Of these initial features, 177,053, or 52%, were immediately eliminated as likely secondary isotope peaks identified by the software system. The 163,938 remaining features were then matched at 2.5 ppm against entries in the default positive mode database. The software system was then used to perform screening based on feature retention time and assignment total acyl carbon number. The software system excluded 7,792 features because the retention time fell outside the range expected for the assignment's parent lipid class. An additional 7,733 features were eliminated because the compound assignment did not contain an even total number of acyl carbon atoms; this optional restriction was applied given the known eukaryotic origin of the data. Adduct ion hierarchy screening was then applied to the remaining 52,337 features. Application of this final orthogonal filter yielded a dataset containing 2,056 compound assignments; these assignments represented 1,969 unique parent compounds (Table 2).

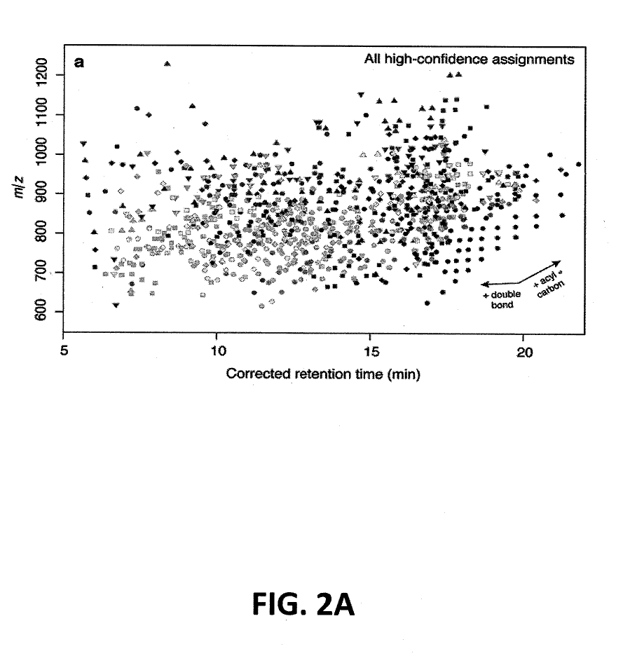

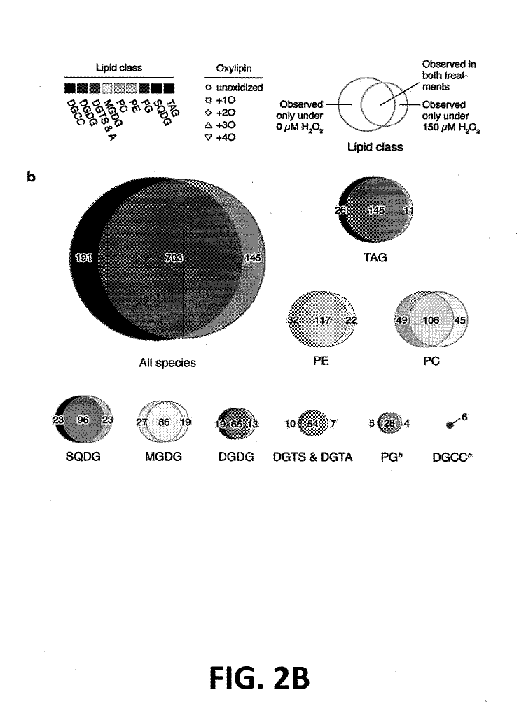

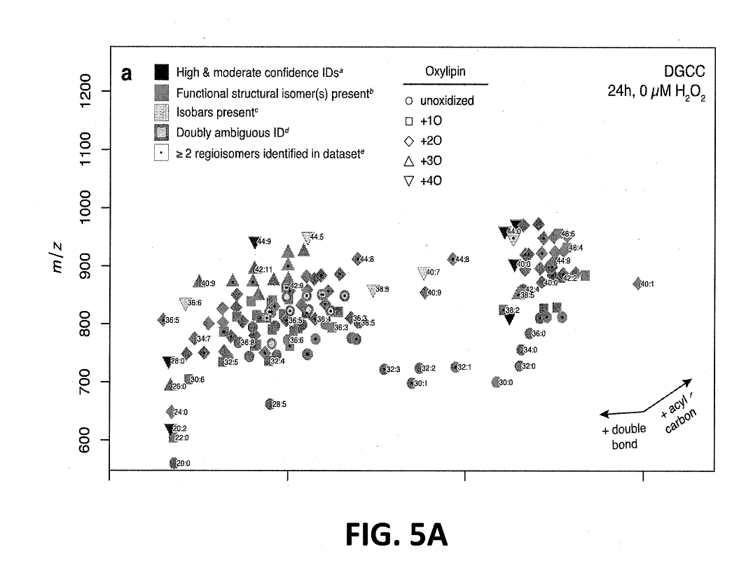

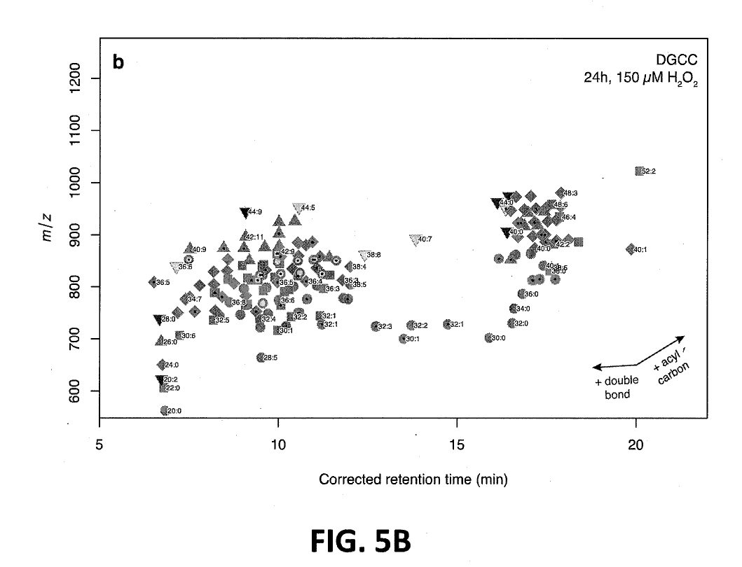

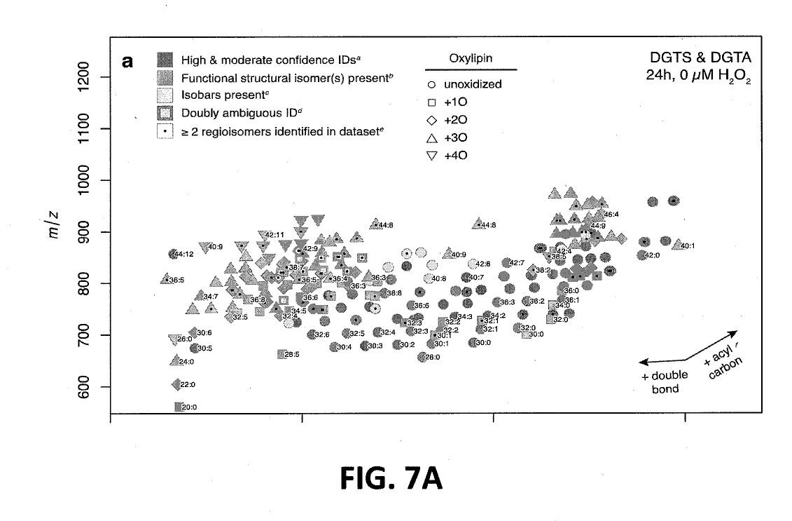

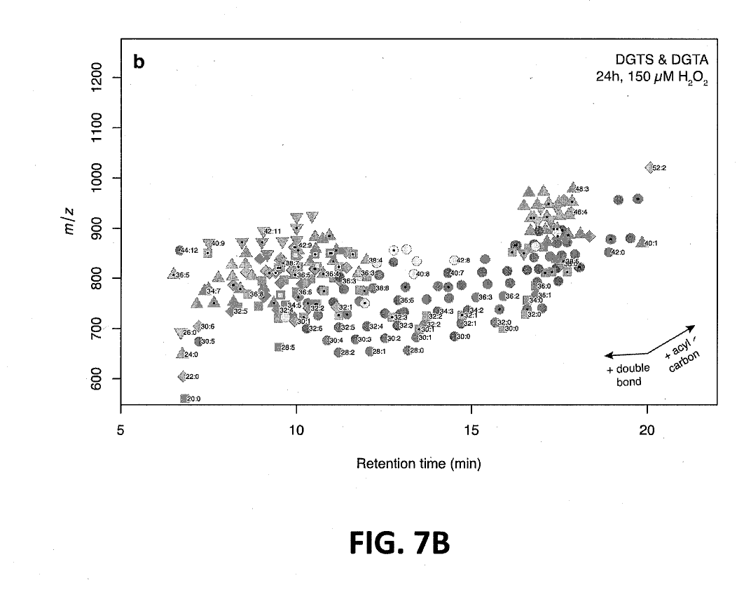

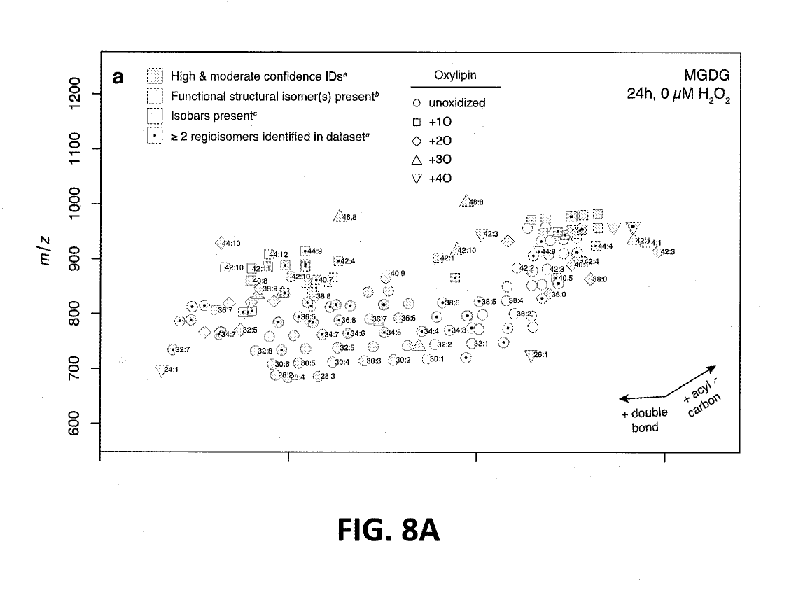

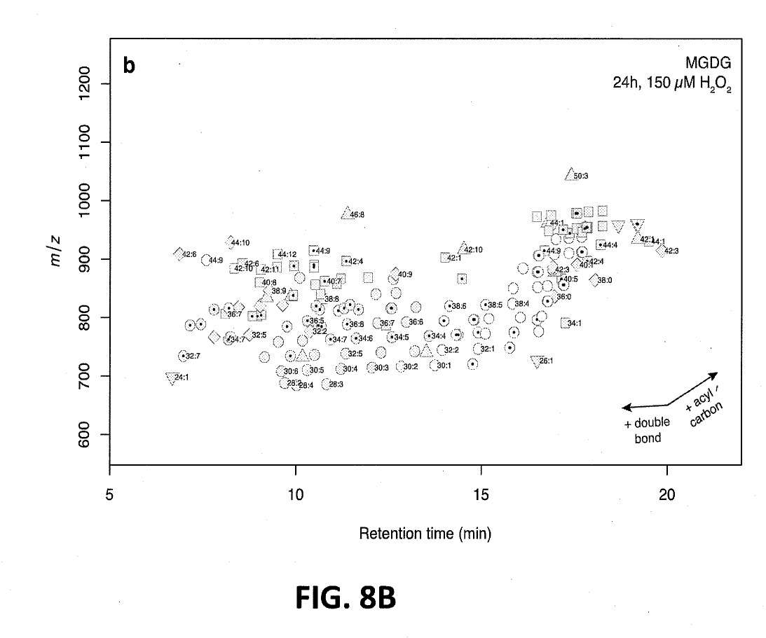

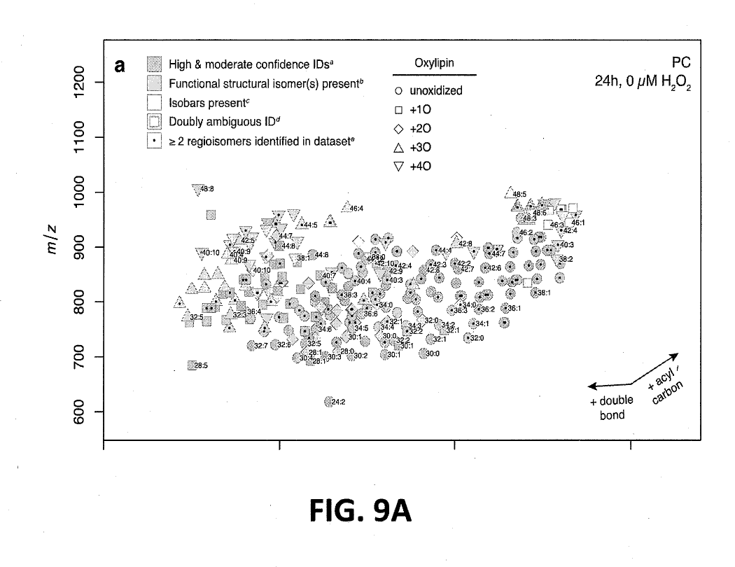

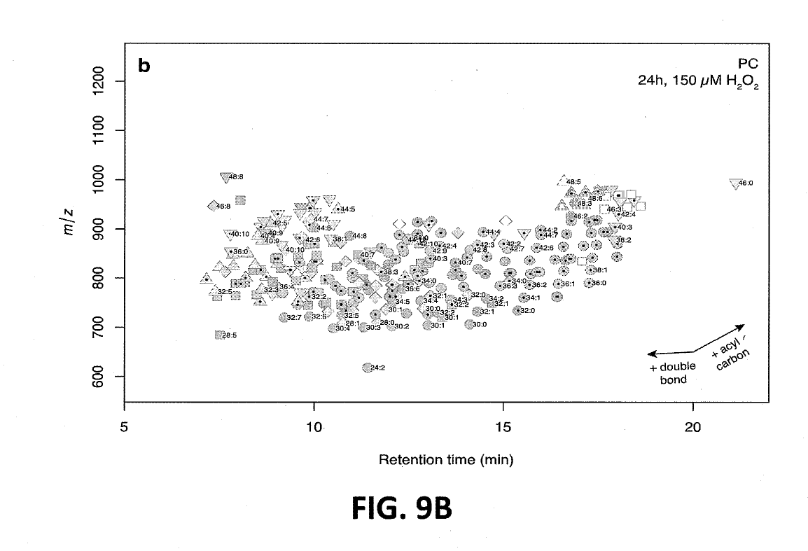

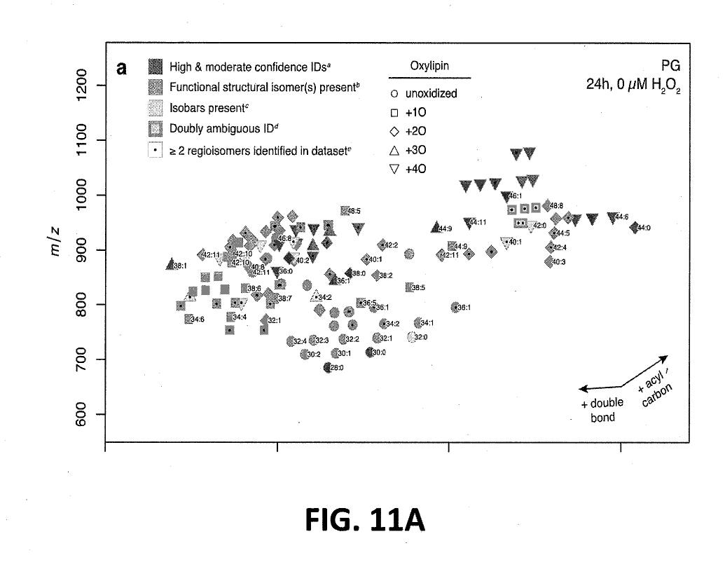

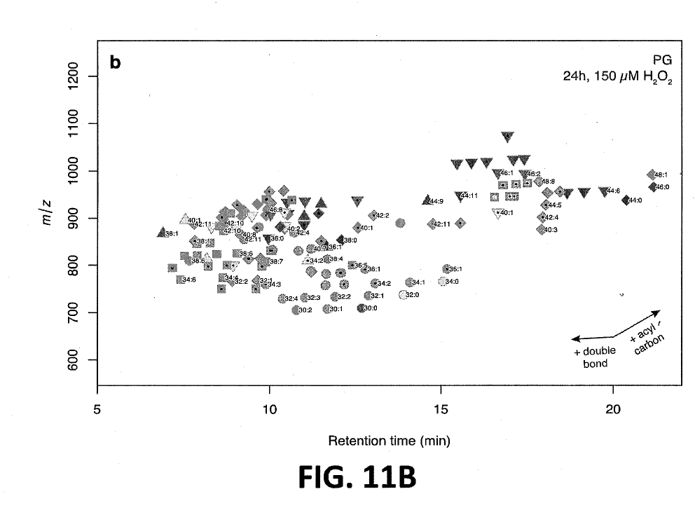

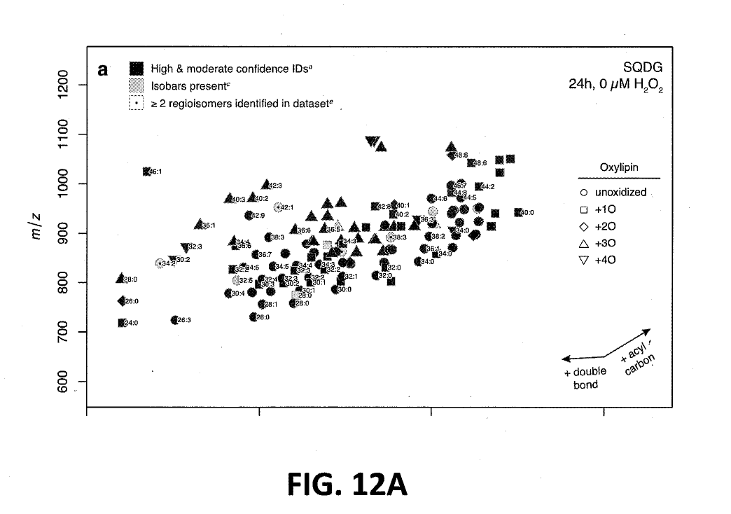

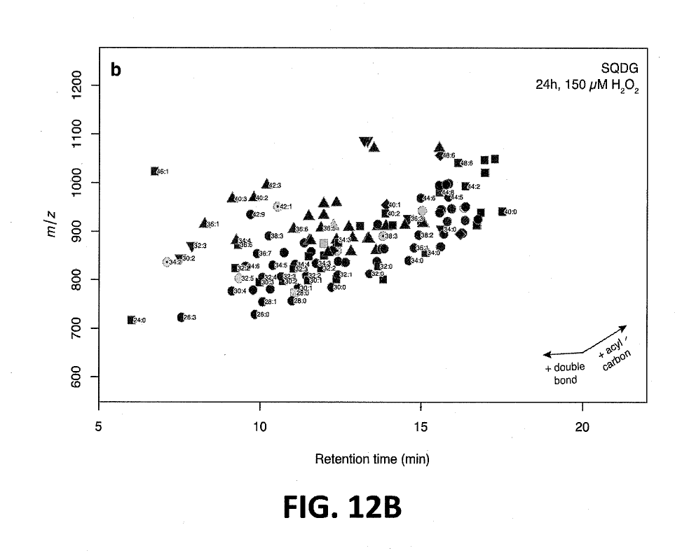

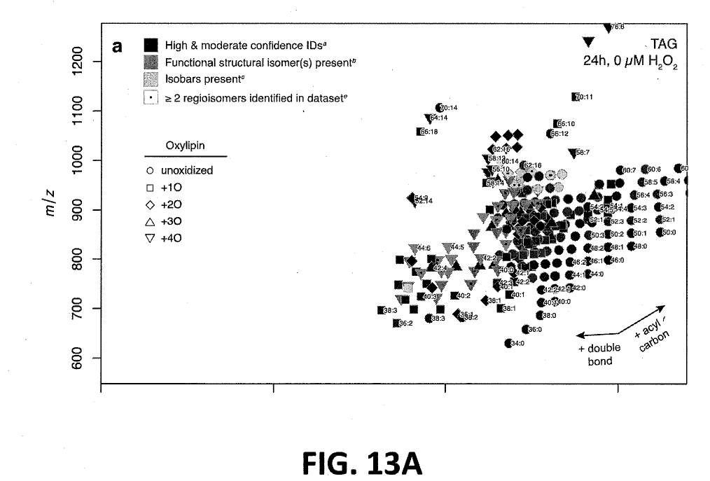

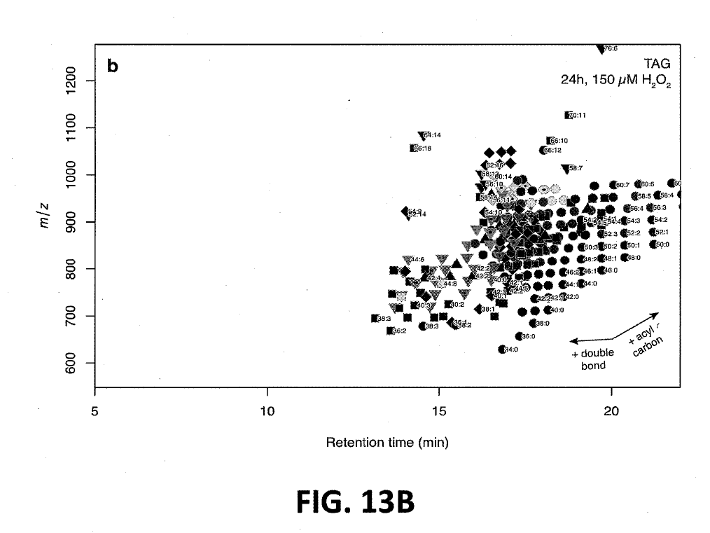

The identities of 1,163, or 57%, of these final database assignments were unique within the scope of the database, meaning the underlying features were matched in the final dataset to only one possible parent compound. 1,149 of these assignments were either IPL, ox-IPL, or TAG as shown in FIG. 2A; the remainder were photosynthetic pigments. 1,056 of these identifications were classified as “high confidence,” indicating that the distribution of adducts present in the constituent features perfectly satisfied the adduct hierarchy rules (FIG. 2A and symbols with darkest tones in FIGS. 5A-13B); these were used in the analysis below. In FIG. 2A, all IPL, ox-IPL, and TAG identified in the P. tricornutum dataset with high confidence (N=1039; figure excludes pigments). The geometric symbols represent types of oxylipin: unoxidized (circle), at least 10 (square), at least 20 (diamond), at least 30 (up triangle) and greater than 40 (down triangle). In FIG. 2B, distribution by lipid class of high-confidence assignments made in the 0 and 150 μM H₂O₂ treatments at 24 h (N=894 and N=848, respectively). Ellipse size in FIG. 2B reflects the number of compounds identified within each class and treatment. The assignments presented in FIGS. 2A and 2B fully satisfied the LOBSTAHS adduct hierarchy screening criteria (i.e., annotated “C1” or “C2a” according to the logic in FIG. 1C and had no competing assignments, such as possible structural isomers, identified in the dataset. Excluded are those compounds having an odd total number of acyl carbon atoms. General direction of movement within m/z versus RT plot, for a given lipid class and oxidation state. The direction of movement that results from addition or removal of additional oxygen atom(s) varies by lipid class.

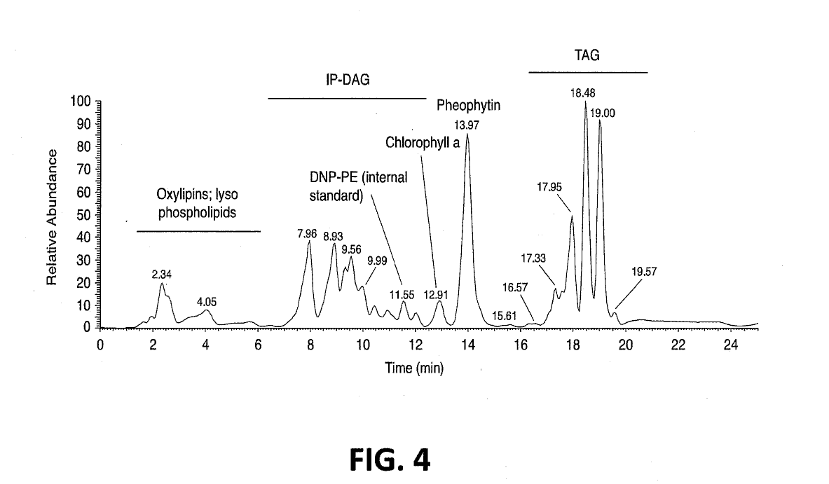

FIG. 4. Is an extracted ion chromatogram (m/z 500-1500; positive ion mode) from a P. tricornutum sample treated with 150 μM H₂O₂. Spectra were acquired under the MS and HPLC conditions described in the text. Text annotations show prominent identifiable features and retention time ranges of some different lipid classes.

### Identification and Annotation of Isomers and Isobars.

The remaining 893 assignments (43.4%) were characterized by some degree of ambiguity, meaning the dataset contained at least one isobar or structural functional isomer of the underlying features (Table 9; symbols with lightest tones in FIGS. 5A-13B). FIGS. 5A-13B show oxidized and intact species of nine classes of lipid identified in the P. tricornutum dataset after 24 hours in (A) the control (0 μM H₂O₂) and (B) 150 μM H₂O₂ treatments according to the present invention. FIGS. 5A-13B show species of, respectively, DGCC, DGDG, DGTS & DGTA, MGDG, PC, PE, PG, SQDG, and TAG. Shading indicates the degree of confidence in the identification, while symbols indicate the degree of oxidation by addition of one or more oxygen atoms. Excluded are those compounds having an odd total number of acyl carbon atoms, according to the reasoning described in the text; this exclusion is an optional, user-electable LOBSTAHS screening feature. Where practical, a text annotation indicates the number of acyl carbon atoms and double bonds in each compound. Data are presented for a single experiment with two technical replicates.

The five shading symbols listed in the upper left of each of the “A” series of FIGS. 5A, 6A, 7A through 13A have the following meanings:
(1) “High & moderate confidence IDs^{a}”, the darkest tones indicate high and moderate confidence IDs (identifications) for which no structural isomers or isobars were detected; these are compounds annotated with codes “C1,” “C2a,” or “C2b” in the LOBSTAHS workflow illustrated in FIG. 1C;
(2) “Functional structural isomer(s) present^{b}”, ≧1 structural isomer of an adduct of this compound is present in dataset (FIG. 1, code C3f);
(3) “Isobars present^{c}”, adduct ion of ≧1 other compound is an isobar of the dominant adduct of this compound; i.e., m/z of the adducts are the 2 ppm match tolerance used in initial assignments (FIG. 1D, code C3c);
(4) “Doubly ambiguous ID^{d}”, ≧1 structural isomer and 1 competing assignment of second type both present; and
(5) “≧regioisomers identified in dataset^{e}”, compounds of which multiple regio-isomers were identified in single sample, indicating possible oxidation of the same parent molecule at different structural positions.
Additionally, the double, angled arrows in the lower right above the phrases “+double bond” and “+acyl^{f} carbon” in each of FIGS. 5A through 13B indicate the general direction of movement within m/z versus RT plot for a given lipid class and oxidation state.

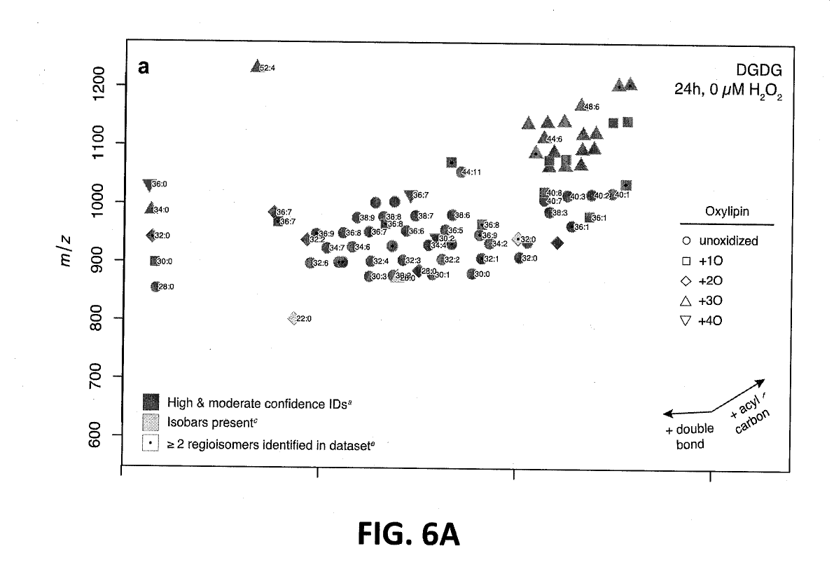

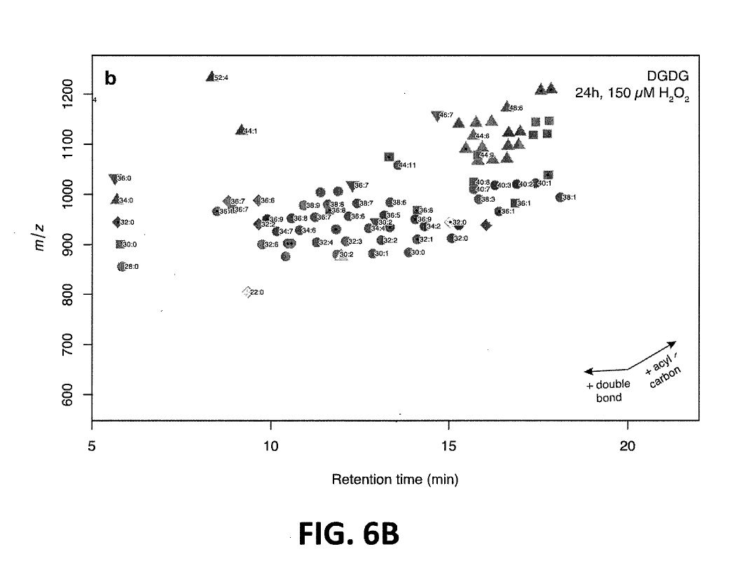

In 752 instances, the dominant adduct of the parent compound was a (functional) structural isomer of the dominant adduct of a different compound assigned from the database (Table 12, first example). In 195 cases, the dominant adduct ion of the parent compound was an isobar of the primary adduct ion of a different compound (Table 12, second example). The 195 ambiguous assignments represented 43.4% of all assignments in the screened dataset, they belonged to just 25% of retained features (27% of peak groups; Table 9). The difference was due to the presence of a small number of features (793) whose 54 assignments were doubly ambiguous, i.e., having both isobars and functional structural isomers (symbols with two-tone shading in FIGS. 5A-13B; Table 12, third example). The number of competing assignments for each identified compound varied largely by lipid class. For example, the software system found no functional structural isomers for compounds identified in several lipid classes: digalactosyldiacylglycerol (DGDG), phosphatidylethanolamine (PE), and sulfoquinovosyldiacylglycerol (SQDG) (FIGS. 6A-6B, 7A-7B and 12A-12B). Doubly ambiguous assignments were confined to only four classes: diacylglyceryl carboxyhydroxymethylcholin (DGCC), diacylglyceryl trimethylhomoserine and diacylglyceryl hydroxymethyl-trimethyl-β-alanine (DGTS & DGTA), phosphatidylcholine (PC), and phosphatidylglycerol (PG) (FIGS. 5A-5B, 7A-7B, 9A-9B, and 11A-11B).

### Annotation of Potential Regioisomers.

The software system also identified regioisomers for 352 unique parent compounds in the P. tricornutum lipidome (Table 9; symbols with black dots in FIGS. 5A-13B). These were instances in which the same assignment was applied to two or more features appearing at different retention times in the same sample. Many of these assignments were oxylipins and ox-IPL, indicating the presence of multiple oxidized isomers of the same parent IPL that could be used as biomarkers for oxidative stress. Without further analysis, the software system is unable to determine whether these isomers represented the oxidation of a fatty acid by the same mechanism at a different acyl carbon position, or instead the presence of different oxidized functional groups that yielded equivalent exact masses (e.g., a dihydroxy-, hydroperoxy-, or α- or γ-ketol acid). The level of identification and annotation provided by the software system supports a wide range of possible molecular structures for each assignment; an example from the dataset is presented in Table 12. The data generated by the software system were consistent with studies in both model plant and animal systems that demonstrated the coexistence of a diversity of ox-IPL with both their parent IPL and smaller, traditional oxylipin degradation products, and thus demonstrating the inventive software system's utility.

### Evaluation of Screening and Identification Performance Using Two Methods.

As a means of validating the accuracy and reliability of the software system's approach, the software system was made to identify and annotate all species present in 5 quality control (QC) samples of known composition that were interspersed randomly with samples from the P. tricornutum dataset prior to analysis on the mass spectrometer (Table 1; Table 8). Table 12 provides examples of isomer and isobar annotation from the P. tricornutum dataset. The samples contained a mixture of authentic IPL standards that has been used extensively in other work. Because the choice of pre-processing software can have a significant impact on feature detection, the software was used in parallel with an alternative software program, MAVEN. In both cases, the inventive software system correctly identified all components of the standard mixture without ambiguity (Table 1; Table 8). As a second means of validation, the software system was run on two independent inventories of the P. tricornutum lipidome and assignments made by the software system were compared. The software system found and identified with high confidence 13 of the 16 most abundant IPL and TAG species in one inventory, and nearly all in the other. This additional tests further proves the utility of the inventive software system.

Resilience of Core P. tricornutum Lipidome under Oxidative Stress.

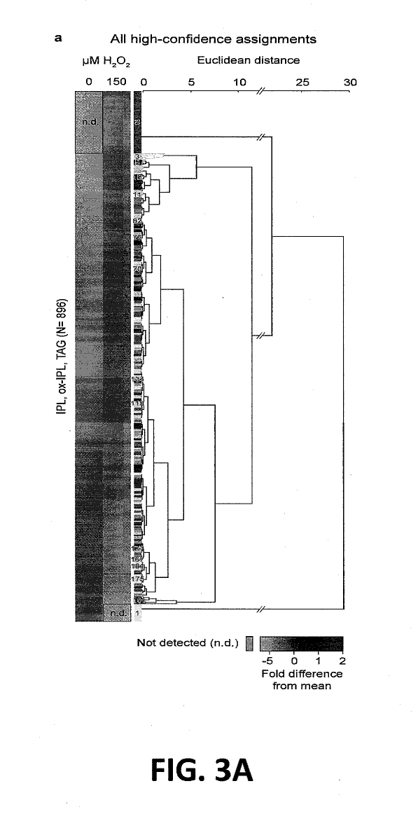

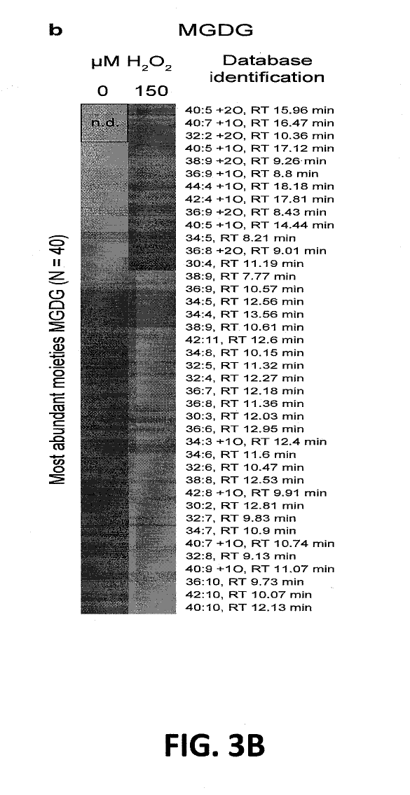

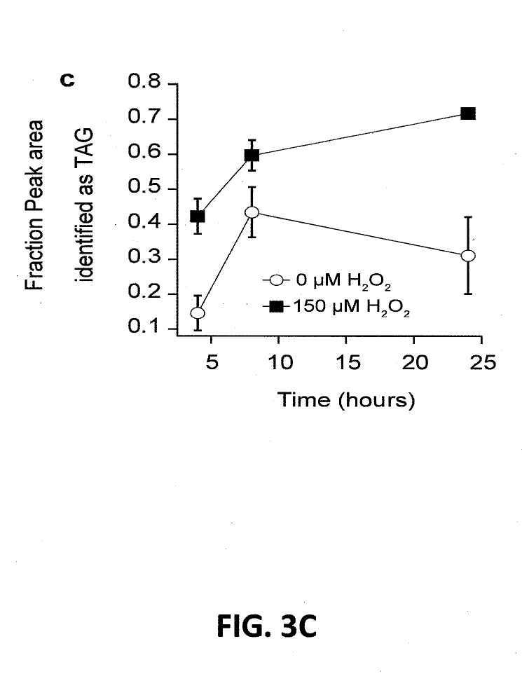

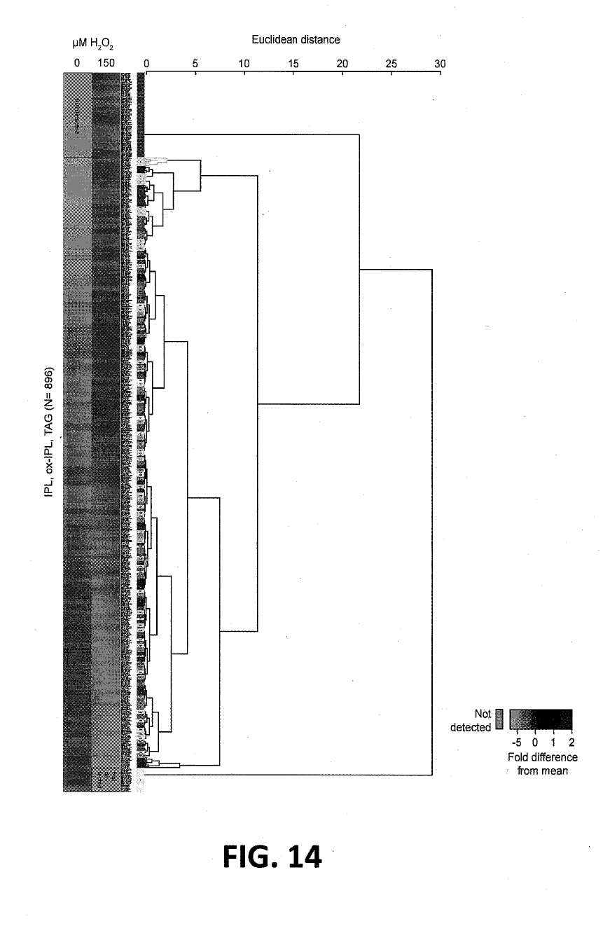

Evidence of the effect of oxidative stress on the lipidome of P. tricornutum was observed through comparison of compounds identified in 0 and 150 μM H₂O₂ treatments at 24 h (FIGS. 2A, 2B, 3A, 3B, and Table 11). That the two treatments produced only subtle differences in molecular diversity (FIG. 2B) suggests much of the core lipid inventory remained robust to the imposed oxidative stress. The vast majority of the 949 oxidized and unoxidized lipid moieties the software system identified in the healthy organism (879, or 92.6%) could still be identified in the lipidome of the stressed cultures (FIG. 2B). On the basis of peak area, oxidized lipid moieties accounted for 5-7% of the P. tricornutum lipidome across nearly all treatments and timepoints. Based on the relatively consistent size of this oxidized lipid fraction and its persistence in even the 0 μM H₂O₂ treatment, it can be thought of as a quantitative constraint on the baseline level of lipid peroxidation associated with metabolic processes in photosynthetic organisms. FIGS. 3A-3C show remodeling of the Phaeodactylum tricornutum lipidome after 24 h, as visualized from data analyzed with the software system. FIG. 3A is a heatmap showing relative abundances across two treatments (0 and 150 μM H₂O₂) of all IPL, ox-IPL, and TAG identified with high confidence. Each row (N=896) represents a different compound identified from the database; FIGS. 14A-B contains an expanded version of the plot that includes labels for each individual compound. FIG. 3B shows heatmap detail, showing changes in the most abundant (N=40) moieties of monogalactosyldiacylglycerol (MGDG), a lipid typically localized to the chloroplast. FIG. 3C Fraction of total peak area identified as triacylglycerol (TAG) at three timepoints during the experiment. Error bars are ±SD of two replicates. In FIGS. 3A and 3B, shading shows the relative abundance of each compound as a fold difference of the mean peak area observed in that treatment from the mean peak area of the compound observed across all treatments. Dendrogram clustering and group definitions were determined by similarity profile analysis. The numbers and identities of the components assigned to each group in FIG. 3A are given in Table 7 and FIGS. 14A-14B. Solid black lines in the dendrogram indicate branching that was statistically significant (P≦1.01).

As noted above, FIGS. 14A-14B are an expanded version of the heatmap in FIG. 3A, showing remodeling of the Phaeodactylum tricornutum lipidome after 24 h. Heatmap shows relative abundances across two treatments (0 and 150 μM H2O2) of all IPL, ox-IPL, and TAG identified by the software system with high confidence. Each row (N=896) represents a different compound identified from the database. Heatmap shading shows the relative abundance of each compound as a fold difference of the mean peak area observed in that treatment from the mean peak area of the compound observed across all treatments; grey shading indicates the compound was not observed. Dendrogram clustering and group definitions were determined by similarity profile analysis of lipidome components based on changes in relative peak area across treatments; methodological details are described in the Supporting Information text. To facilitate visualization, each group was randomly assigned a different color; group numbers corresponding to those in Table 10 are also indicated. Solid black lines in the dendrogram indicate branching that was statistically significant (P≦0.01). Table 10 lists the numbers and identities of the components assigned to each group. Data are presented for a single experiment with two technical replicates.

### Differences in Degree of Remodeling Between Lipid Classes and Functional Groupings.

The software system's similarity profile analysis of the scaled data was used to place the annotated features into 181 groups of components which clustered significantly according to their behavior (FIGS. 3A-3C and FIGS. 14A-14B). The components of each group are given in Table 10. The up- and downregulation of lipidome components under oxidative stress were further examined by dividing potential biomarkers into classes based on their molecular headgroups (Table 11). This portioning allowed the software system to examine class-specific differences in the number of acyl carbon atoms, acyl carbon-to-carbon double bonds, and oxidation states (i.e., additional oxygen atoms) of component lipids under the two treatments. Differential expression of chemical properties within several classes (FIGS. 3A-3C; Table 11) suggested the P. tricornutum lipidome was remodeled in subtle but pervasive ways.

### Fatty Acid Chain Elongation is an Apparent Response to Oxidative Stress in the Chloroplast.

Oxidative stress appeared to induce elongation of fatty acids throughout the P. tricornutum lipidome (Table 11). Lipid moieties upregulated by oxidative stress had longer fatty acid chains than those that were downregulated. The greatest breadth of structural change was in monogalactosyldiacylglycerol (MGDG), a lipid typically localized to the chloroplast (Table 11; FIG. 2B). Moieties of MGDG upregulated in the 150 μM H₂O₂ treatment had significantly longer fatty acid chains and were more oxidized than those downregulated under oxidative stress; oxidation and elongation were also accompanied by a statistically significant decrease in acyl chain unsaturation (Table 11). The MGDG moieties responsible for these shifts in class structural properties were confined largely to groups 1, 2, 4, 5, 7, 9, 12, 166, 167, and 180 in our similarity profile analysis (FIGS. 3B and 14A-14B; Table 10). Lipid oxidation has been previously linked in the diatom Skeletonema costatum to lipolytic cleavage of MGDG and phospholipids within the chloroplast, resulting in oxylipin production from free fatty acids. While intact oxidized MGDG species have not been previously observed in algae, ox-MGDG have been documented in terrestrial plants upon wounding.⁸ The production of ox-IPL in Arabidopsis thaliana may be a means of binding ROS within the cell membrane to limit damage elsewhere.

### Significant Enrichment Observed in TAG.

Whereas the impact of oxidative stress within most lipid classes was confined to relatively modest changes in structural properties, treatment with 150 μM H₂O₂ induced a very significant enrichment in the fraction of peak area the software system identified as triacylglycerols (TAG; FIG. 3C). Enigmatically, the TAG moieties upregulated in the 150 μM treatment were significantly less oxidized than those downregulated. The growth of this chemically reduced TAG pool may be evidence of enhanced de novo production of un-oxidized TAG as a response to oxidative stress. Increased TAG synthesis is a known response to nutrient starvation in virtually all algae, including in P. tricornutum. While increased TAG production in algae has not been previously linked directly to oxidative stress, increased production has been observed as a response to viral infection in the haptophyte alga Emiliania huxleyi.

### Example 2

Conversion of .raw Data Files to .mzXML Format.

After acquiring data from the mass spectrometer, the software system converts all Thermo .raw files in a given dataset to the open-source .mzXML format, which is used by many chromatographic alignment and peak picking applications. It then converts the profile-mode mass spectral data in each file to a series of centroids. Finally, the software system automates the extraction of the positive and negative ion mode full scan events from each sample into separate files. In the Exactive instrument configuration described above, the full scan events from the two ion modes appeared in each data file as the first and third scan events at each time point, respectively. (The second and fourth scan events at each time point were the positive and negative mode AIF scans.) The extraction and separation of scans from the two ion modes was necessary to accomplish subsequent analysis using the pipeline.

### Sample Injection, Chromatography and ESI Source Settings.

20 μL injections of sample were made onto a C8 Xbridge HPLC column (particle size 5 μm, length 150 mm, width 2.1 mm; Waters Corp., Milford, Mass., USA). Eluent A consisted of water with 1% 1M ammonium acetate and 0.1% acetic acid. Eluent B consisted of 70% acetonitrile, 30% isopropanol with 1% 1M ammonium acetate and 0.1% acetic acid. Gradient elution was performed with the following program (total run time 30 min) at a constant flow rate of 0.4 mL min⁻¹: 45% A for 1 min to 35% A at 4 min, then from 25% A to 11% A at 12 min, then to 1% A at 15 min with an isocratic hold until 25 min, and finally back to 45% A for 5 min column equilibration. ESI source settings were: Spray voltage, 4.5 kV (+), 3.0 kV (−); capillary temperature, 150° C.; sheath gas and auxiliary gas, both 21 (arbitrary units); heated ESI probe temperature, 350° C.

### Mass Spectrometer Acquisition Settings.

Mass data were collected on a ThermoFisher Exactive Plus Orbitrap instrument in full scan (FS) and all-ion-fragmentation modes (AIF) while alternating between positive and negative ion modes. A scan range of 150-1500 m/z was used for all modes in sequence (FT MS positive full scan, FT MS positive AIF, FT MS negative full scan, and FT MS negative AIF, respectively). The S-lens RF level was set to 85.00. Mass resolution was set to the maximum possible value of 140,000 (FWHM at m/z 200) for both FS and AIF. This mass resolution setting corresponded to an observed resolution of 75,100 at the m/z (875.5505) of the internal standard, DNP-PE. The observed resolution at m/z 1269.0952, that of the compound in the screened dataset with the highest molecular weight (TAG 76.6+4O), was 41,100. Using these settings, 8 and 14 MS scans across a typical peak were obtained.

### Procedures Used for Weekly and Real-Time Calibration of the Exactive.

The mass spectrometer was calibrated weekly in both positive and negative ion modes by infusing calibration mixes available from ThermoFisher Scientific. Low-level eluent contaminants were also utilized as lock masses, providing real-time recalibration; C16:0 (255.23295) and C18:0 (283.26425) fatty acids were used in negative ion mode, while a polysiloxane (536.16537) and phthalate (391.28429) were used in positive ion mode. At least one of the lock masses was found during each positive and negative full scan event.

Script for Pre-Processing Data in xcms and CAMERA.

In this embodiment, the software system accepts data preprocessed by the xcms and CAMERA scripts (utilized in the R computer program) using the script “prepOrbidata.R.” The user can further modify the script as necessary. The R package IPO was used to optimize settings for xcms and CAMERA, obtaining the parameter values given in Table 7. We used these parameter values to obtain the results presented in the text.

### Determination of Retention Time Window Data.

The retention time (RT) window data in Table 6 were obtained primarily from authentic standards for representative compounds of each parent lipid class under the chromatographic conditions described in above. Observations of various lipids in environmental samples allowed the consideration of additional species. While the software system applies the retention time data contained in Table 6 as a default, detailed instructions and an example data table are included in the onboard documentation for use with retention time data for other chromatographic methods. As for the adduct ion hierarchy data, retention time data for ox-IPL are inherited from the unoxidized parent molecule. By default, the software system expands the retention time window for each lipid class by 20% of its given width to account for (1) shifts in retention time that may occur during chromatographic alignment with xcms and (2) slight variations in retention time that distinguish the different positional (i.e., regio-) isomers of the same parent lipid. This window can be narrowed or expanded with user input.

Analysis of Positive Ionization Mode P. tricornutum Data Using Xcms, CAMERA, and One Embodiment of the Software System.

To examine the effect of oxidative stress on the P. tricornutum lipidome, the software system workflow in FIGS. 1A-1D was applied to a dataset assembled from only positive mode data files. The analysis was confined to the positive mode data because intact polar lipids (IPL), the primary targets of both reactive oxygen species (ROS) and lipoxygenase-mediated enzymatic transformations induced by H₂O₂, are most amenable to analysis in positive ion mode. The specific workflow and parameter values applied to the dataset in xcms, CAMERA, and the software system are given in Table 7. All three optional filters to the data were applied in this example of the inventive software system.

### Choice of Matching Tolerance.

To account for variability in performance expected from natural samples, a 2.5 ppm mass uncertainty tolerance was used when matching against the databases. This tolerance was one order of magnitude more conservative than the 0.22 ppm mass uncertainty observed with authentic standards (Table 1 and Table 8), yet considerably more restrictive than the various default standards used for matching in other recently introduced metabolomics applications. When combined with HPLC separation and the high mass resolution of the Exactive, the 2.5 ppm tolerance still allowed for the assignment of distinct identities to isobaric masses.

Statistical Analysis and Visualization of the P. tricornutum Lipidome.

The software system workflow is designed to facilitate examination of relative changes in the abundances of lipids in a given dataset, not to enable absolute quantification of specific analytes or direct comparisons between datasets. With this in mind, the annotated output from the software system was used to calculate the relative abundances of P. tricornutum lipidome constituents present in the 0 and 150 μM H₂O₂ treatments at 24 h. The analysis was performed as follows:
1. The processed dataset was extracted using the “PtH2O2_mz-rt_plots.R” script and a subset of “high confidence” assignments to be used in all subsequent analyses (i.e., assignments annotated with codes C1 or C2a and having no identified structural isomers or isobars; FIG. 2A and symbols with darkest tones in FIGS. 5A-13B). The remaining putative assignments were classified as “moderate confidence,” indicating that the underlying features satisfied the hierarchy rules fundamentally yet imperfectly (symbols with lighter tones in FIGS. 5A-13B). The peak areas of the high confidence assignments were normalized to data for an internal standard (DNP-PE). The analysis was further restricted to only those compounds still present in two or more samples.
2. Using the script “PtH2O2_heatmap_sigclust.R,” peak areas of the remaining assignments were then scaled using a level approach according to⁶. Each peak area, xᵢ, was divided by the average peak area of that compound across the dataset, x_{avg}, to obtain a normalized peak area, \tilde{x}ᵢ:

$${\tilde{x}}_{i} = \frac{{x}_{i}}{{x}_{avg}}$$

Values of \tilde{x}ᵢ were then used in the steps described below to represent the relative abundances of the compounds present in each sample.
3. When the same compound appeared in duplicates of the same experimental treatment, values of \tilde{x}ᵢ were averaged. Since subsequent statistical analysis required log₂ transformation of the data and many hierarchical clustering functions cannot use values of 0 or “NA” as inputs, all values of \tilde{x}ᵢ equal to 0 were replaced with 10⁻⁶.
   In this example, the heatmaps and dendrogram in FIGS. 3 and 14 were then generated from this subset of relative abundances using the R packages gplots and clustsig. In other embodiments, the inventive software system creates heatmaps and dendrograms. Similarity profile analysis was then used to cluster the molecular species in the subset according to their degree of covariation. The groups of lipidome components identified by this similarity profile analysis are presented in Table 10. Following this analysis, the heatmaps in FIGS. 2 and 14 was reordered and the dendrogram was rotated such that the order of compounds in both figures is from most upregulated in the 150 μM H₂O₂ treatment to most downregulated. The groups from the similarity profile analysis (Table 10) were categorized by the general response of their components to H₂O₂ treatment at 150 μM H₂O₂: 101 groups contained a total of 562 compounds more abundant in the 150 μM treatment (i.e., upregulated), 70 groups contained a total of 308 compounds less abundant in the 150 μM treatment (i.e., downregulated), and 11 groups together contained 26 components whose abundance was not significantly different between the two treatments (P≦0.01).

Mass spectrometry workflow is enhanced according to the present invention by high-throughput annotation and putative identification of ionizable molecules in high-mass-accuracy HPLC-MS data. Orthogonal, rules-based screening criteria are utilized based on adduct ion formation hierarchy patterns and other properties to accurately identify compounds. A confidence value may be generated for each assignment, and a user interface such as a display screen or a printer may present screening results such as tables, graphs, diagrams or lists to a user.

Reference throughout this specification to “one embodiment,” “an embodiment,” “one construction”, or similar language means that a particular feature, structure, or characteristic described in connection with the embodiment is included in at least one embodiment. Thus appearances of the phrase “in one embodiment,” “in one construction,” and similar language throughout this specification may, but do not necessarily, all refer to the same embodiment.

Although specific features of the present invention are shown in some drawings and not in others, this is for convenience only, as each feature may be combined with any or all of the other features in accordance with the invention. While there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature.

It is the intention, therefore, to be limited only as indicated by the scope of the claims appended hereto. Other embodiments will occur to those skilled in the art and are within the following claims.

The invention claimed is:

1. A method of screening a plurality of molecules in datasets obtained from mass spectroscopy, the method comprising:
(a) selecting and receiving at least one dataset of mass spectral data;
(b) selecting customizable m/z mass tolerance peaks to assign initial compound assignments from at least one adduct ion hierarchy database for at least one compound having a parent molecule; and
(c) applying adduct ion hierarchy screening to at least a portion of the database, wherein selected dataset features are tested to determine if they represent the most abundant expected adduct of the parent molecule class and if the expected adduct assignment hierarchy are present in the dataset.

2. The method of claim 1 further including pre-processing the dataset including aggregating the dataset into subsets based on at least one feature.

3. The method of claim 2 further including applying a screening criteria to identify and remove secondary isotypes in the subsets.

4. The method of claim 2 further including applying a screening criteria of retention time to at least one subset of the database based on a relationship of retention time of the dataset features as compared with a predefined retention time window of the compound assignment's parent molecule class.

5. The method of claim 4 further including applying a screening criteria to identify and annotate isomer and isobars in the subset.

6. The method of claim 4 further including assigning one or more identifications and annotations to each feature in the subset.

7. The method of claim 1, wherein preprocessing the dataset includes at least one of feature detection, retention time correction, peak grouping, m/z, secondary isotope identification, and a combination thereof.

8. The method of claim 1, wherein the feature is selected from retention time, acyl carbon number, peak, and a combination thereof.

9. The method of claim 1, wherein the first screening criteria includes identifying and excluding secondary isotopes.

10. The method of claim 4 further including applying a screening criteria to the subset to exclude one or more specific molecules, specific chemical moieties or molecules containing a specific number of one or more chemical moieties, in a customizable manner.

11. A system for screening a plurality of molecules in mass spectroscopy datasets, the system comprising a processor programmed to execute the method of claim 1, and a user interface to present screening results to a user.

12. A computer program for screening a plurality of molecules in mass spectroscopy datasets, the program comprising the method of claim 1, wherein said program is executed on a computer device.

13. The method of claim 4, further including formatting the screened subset such that it will be analyzable by additional software on a computer device.

14. The method of claim 4, further including performing statistical analysis on the subset after adduct ion hierarchy screening is applied.

15. The method of claim 4, further including exporting the screened subset to a common file format readable by additional software on a computer device.

16. The method of claim 4 wherein the method annotates the resulting subset with codes demarking the degree to which the assignment complies with the hierarchy screening criteria.

17. The method of claim 1 wherein at least one adduct ion hierarchy database is generated in at least one of a positive ion mode and a negative ion mode.

18. The method of claim 1 further including generating the dataset utilizing at least one additional chemical that is added to an eluent to which the molecules are exposed at least prior to the mass spectroscopy.

19. The method of claim 1 further including preparing at least one adduct ion hierarchy database, and selecting that database for screening the dataset.

20. The method of claim 1 further including generating adduct ion hierarchy databases from empirical data produced from standardized parent molecules that have undergone ionization and measurement by mass spectrometry.

21. The method of claim 17 further including ranking the databases by adduct ion ranking.

22. The method of claim 1 wherein the mass spectrometry data is selected to include at least one of liquid chromatography-mass spectrometry data, gas chromatography-mass spectrometry data, Fourier transform mass spectrometry data, direct infusion mass spectrometry data, capillary electrophoresis mass spectrometry data, ion mobility shift mass spectrometry data, desorption electrospray ionization mass spectrometry data, nanostructure initiator mass spectrometry or matrix assisted mass spectrometry data.

23. The method of claim 1 further including generating a confidence value for the identifications assigned to each feature.
